(19) United States
(12) Patent Application Publication
(10) Pub. No.: US 2026/0318898 A1
(43) Pub. Date: Oct. 15, 2026

(54) MEASURING ION CONTENT IN SUBTERRANEAN AQUEOUS FLUIDS

(71) Applicant: Schlumberger Technology Corporation, Sugar Land, TX (US)

(72) Inventors: Emma Irene Corcoran, Cambridge (GB); Debora Campos de Faria, Cambridge (GB)

(21) Appl. No.: 19/648,444

(22) Filed: Apr. 15, 2026

(30) Foreign Application Priority Data
Mar. 30, 2025 (GB) 2506614.3

(51) Int. Cl. G01N 33/28; G01N 27/02

(52) U.S. Cl. CPC G01N 33/2823; G01N 27/028

(57) ABSTRACT

Rapid determination of concentration of chloride ions, or bromide ions in an aqueous fluid which is at a subterranean location or has come to the surface from a subterranean location is carried out by a voltametric system without requiring separation from other ions. The method may include determining whether measured peak current lies outside a predetermined range and if so, diluting the sample by a known amount or adding a known amount of the analyte so as to obtain a measured value within the predetermined range. Initial separation of aqueous fluid from solids may be achieved using a rotary drum with a filter material at its surface.

### CROSS REFERENCE TO RELATED APPLICATIONS

This application claims priority to GB Patent Application No.: 2506614.3, entitled “Measuring ion content in subterranean aqueous fluids”, which was filed on Apr. 30, 2025, and is incorporated herein by reference in its entirety.

### FIELD

This disclosure is concerned with determining the concentration of halide ions, which may be chloride or bromide ions, in an aqueous solution. The method and apparatus of this disclosure may be used for determining concentration in water or aqueous solution which is at a subterranean location, or which has come from a subterranean location.

### BACKGROUND

Drilling fluid (often referred to as drilling mud) may be an emulsion with an aqueous continuous phase. It is common for it to include potassium chloride to provide or enhance salinity, as well as other salts. As a wellbore is drilled, the drilling fluid is in contact with the underground geological strata, so that water may move to or from the strata by osmosis, and salts in the underground water held in porous strata may also enter the drilling fluid. Thus, returning drilling fluid is likely to contain mixtures of anions and mixtures of cations. Water from other subterranean sources may also contain mixtures of dissolved salts and so contain mixtures of anions and mixtures of cations.

It is routinely required to analyze drilling fluid as it returns to the surface from a wellbore which is being drilled. American Petroleum Institute (API) standard methods are able to determine concentrations of individual analyte ions but mostly involve titrations for individual analytes and require various reagents to be added to samples before titration takes place. Because a chloride salt is commonly included to provide salinity, the concentration of chloride ions is a significant parameter to measure. Also, it may be useful to carry out measurements downhole to determine concentrations of halide ions in an aqueous fluid encountered downhole, such as aqueous solution in the geological formation around a wellbore. Here also the aqueous solution may contain a mixture of anions. A number of methods of measurement differing from the API standard methods have been proposed. These generally require separation of the various anions present before measuring their individual concentrations.

One such method is ion-chromatography in which a fluid sample is separated on an ion-exchange column, so that the ionic species are eluted in sequence and their concentrations can be determined as they flow through a cell in which conductivity is measured. Ion-chromatography methods have been described in European patent EP0282231 and Society of Petroleum Engineers papers SPE23076 and SPE25703.

Another method which has been put forward more recently and again involves separation of the ionic species present before making quantitative measurement is capillary electrophoresis as disclosed in U.S. Pat. Nos. 9,976,417 and 11,179,114.

A different approach for separating one ion to be measured from other ions present in a solution is to use a membrane which is permeable to a chosen ionic species but impermeable to others. There is considerable scientific literature concerned with ion-specific electrodes for various ions, including halide ions. These electrodes are generally used to determine concentration by a potentiometric method. For example, Rechnitz and Kresx describe chloride-specific and bromide-specific electrodes in “Potentiometric Measurements with Chloride-Sensitive and Bromide-Sensitive Membrane Electrodes”, Analytical Chemistry, Vol 38, Pages 1786-1788 (1966).

Endeavors to achieve good selectivity of ion-selective electrodes have led to electrodes of elaborate construction and/or requiring unusual materials, as in Li et al “An all-solid-state polymeric membrane chloride ion-selective electrode with nanowires poly(3,4-ethylenedioxythiophene) as solid contact”, Int. J. Electrochem. Sci., Vol 12, Pages 11715-11727 (2017) and DiFranco et al., “A highly-selective chloride microelectrode based on a mercuracarborand anion carrier”, Sci Rep 9, 18860 (2019).

Kaland et al in “Use of an Ion-Selective Electrode for Determination of Free Chloride Ions in Water-Based Drilling Fluids”, SPE Drilling Engineering, October 1986, Page 365 (also Society of Petroleum Engineers paper SPE 15147) used a commercially available chloride specific electrode in a potentiometric method but mentioned a need to avoid ions which would interfere with the response to chloride.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

The inventors have now found that voltammetry is able to provide a measurement of chloride and/or bromide ion concentration without requiring that the chloride or bromide ions are separated from other ions which are likely to be present. This may be used below ground, or when fluid from an underground location has been brought to the surface. In particular, this may be useful in circumstances where drilling fluid has returned to the surface or where fluid has come to the surface from a subterranean location in some other way. Being able to use voltammetry without previous separation of individual ions is advantageous for monitoring chloride (and/or bromide) concentration in aqueous drilling fluid as it returns to the surface, because it avoids delay required for a separation procedure such as chromatography, and is not requiring the skill needed for manual titration and can give a result quickly after a sample is collected.

In a first aspect this disclosure provides a method of determining concentration of analyte ions, which are chloride and/or bromide ions, in an aqueous fluid which is at a subterranean location or which has come to the surface from a subterranean location. The fluid may be a drilling fluid which has returned to the surface. The method comprises: providing a voltammetric system comprising a working electrode, a reference electrode and a counter electrode and electronic circuitry configured to apply varying potential, relative to the reference electrode, to the working electrode and measure and record the current through the working electrode, contacting a portion of the aqueous fluid with the electrodes, with the counter electrode's surface area exposed to the fluid being at least twice the working electrode's surface area exposed to the fluid, and operating the voltammetric system to apply potential to the working electrode across a range in which the applied potential causes an electrochemical reaction in which analyte ions are liberated into or removed from the solution at the working electrode and the current for that reaction passes through a peak value, and to record the current at the peak, or record the current over a range of potential including the peak, as indicative of concentration of the analyte ions in the fluid. Recording current over a range of potential provides a measurement of the charge transferred through the working electrode as the potential is scanned across that range.

A sample of the aqueous fluid may be collected below ground or at the surface as a sample consisting solely of an aqueous solution, for instance if it is a sample of water present in porous subterranean rock or which has come to the surface from such rock. Other collected samples may contain a non-aqueous liquid phase and/or contain suspended solids. For these, a form of the present method may include breaking any emulsion and separating the aqueous fluid from any solids and any non-aqueous liquid. In particular, this may be accomplished when the fluid is water-based drilling fluid which has returned to the surface. Such a drilling fluid may contain a continuous aqueous phase and a discontinuous oil phase with at least one chloride or bromide salt dissolved in the aqueous phase, viscosity enhancing polymer in the aqueous phase and insoluble particulate material suspended in the aqueous phase.

The method of this disclosure can be employed over a useful range of concentration where the recorded current is proportional to the concentration of the analyte ions. Moreover, the working range can be extended to higher concentrations of analyte ions by carrying out the voltammetry on sample fluid which has been diluted by a known amount. The concentration of analyte ions in the original sample can then be calculated from the concentration determined by voltammetry and the known extent of dilution. Conversely, the working range can also be extended to lower concentrations by adding a known amount of a chloride, or a bromide, salt to increase the concentration by a known amount, and the concentration of analyte ions in the original sample can then be calculated from the result of voltammetry and the known increase in concentration.

In a second aspect, this disclosure provides a voltammetric system comprising a working electrode, a reference electrode and a counter electrode with an exposed surface area which is at least twice the size of the exposed surface area of the working electrode and electronic circuitry configured to apply varying potential, relative to the reference electrode, to the working electrode and measure and record the current through the working electrode, the voltammetric system being operable to apply potential to the working electrode across a range in which the applied potential causes an electrochemical reaction in which analyte ions are liberated into or removed from the solution at the working electrode and the current for that reaction passes through a peak value, and to record the current at the peak, or record the current over a range of potential including the peak, as indicative of concentration of the analyte ions in the fluid.

### BRIEF DESCRIPTION OF THE DRAWINGS

The subject disclosure is further described in the detailed description which follows, in reference to the noted plurality of drawings by way of non-limiting examples of the subject disclosure, in which like reference numerals represent similar parts throughout the several views of the drawings, and wherein:

FIG. 1 is a schematic diagram of a conventional drilling system;

FIG. 2 is a schematic diagram of apparatus for voltammetry;

FIG. 3 shows an electrode assembly;

FIG. 4A shows superimposed electrochemical signals (voltammograms) recorded during cyclic voltammetry on sodium chloride solutions;

FIG. 4B shows results from FIG. 4A and similar data as a plot of peak current against chloride concentration;

FIG. 5A shows superimposed voltammograms recorded during cyclic voltammetry on potassium chloride solutions of various pH;

FIG. 5B shows results from FIG. 5A as a plot of peak current against chloride concentration;

FIG. 6 shows superimposed voltammograms recorded during cyclic voltammetry on solutions containing both sodium chloride and calcium chloride;

FIG. 7 shows superimposed voltammograms recorded during cyclic voltammetry on solutions containing several cations, typical of water from a subterranean location;

FIG. 8 shows superimposed voltammograms recorded during cyclic voltammetry on filtrates from examples of drilling fluid having varying pH;

FIG. 9 shows the oxidative part of voltammograms recorded during cyclic voltammetry on solutions containing a mixture of dissolved salts;

FIG. 10A shows superimposed voltammograms for 1M sodium chloride and 1M sodium bromide:

FIG. 10B shows a plot of peak current against bromide concentration recorded during cyclic voltammetry on sodium bromide solutions;

FIG. 11 shows plots of peak current against sodium chloride concentration obtained using various types of voltammetry;

FIG. 12 shows a disposable electrode assembly;

FIG. 13 is a side view of the assembly fitted to a connector;

FIG. 14A shows superimposed voltammograms recorded during cyclic voltammetry on sodium chloride solutions using a disposable electrode assembly as shown in FIG. 12;

FIG. 14B shows results from FIG. 14A as a plot of peak current against chloride concentration;

FIG. 15 shows separation of solids and liquid with a vacuum drum;

FIG. 16 is a flowchart showing a procedure for determination of chloride content;

FIG. 17 shows a portion of a borehole with a wireline tool for making voltammetric measurement at a subterranean location; and

FIGS. 18, 19 and 20 show electrode assemblies with additional electrodes.

### DETAILED DESCRIPTION

The particulars shown herein are by way of example and for purposes of illustrative discussion of the examples of the subject disclosure only and are presented in the cause of providing what is believed to be the most useful and readily understood description of the principles and conceptual aspects of the subject disclosure. In this regard, no attempt is made to show structural details in more detail than is necessary, the description taken with the drawings making apparent to those skilled in the art how the several forms of the subject disclosure may be embodied in practice. Furthermore, reference numbers and designations in the various drawings indicate like elements.

This detailed description shows various embodiments of the present disclosure and possibilities which may be used. It should be appreciated that features or possibilities described in combination may, where it is practical to do so, be used individually. Also, features or possibilities described in any embodiment may be used in any other embodiment, in so far as it is practical to do so.

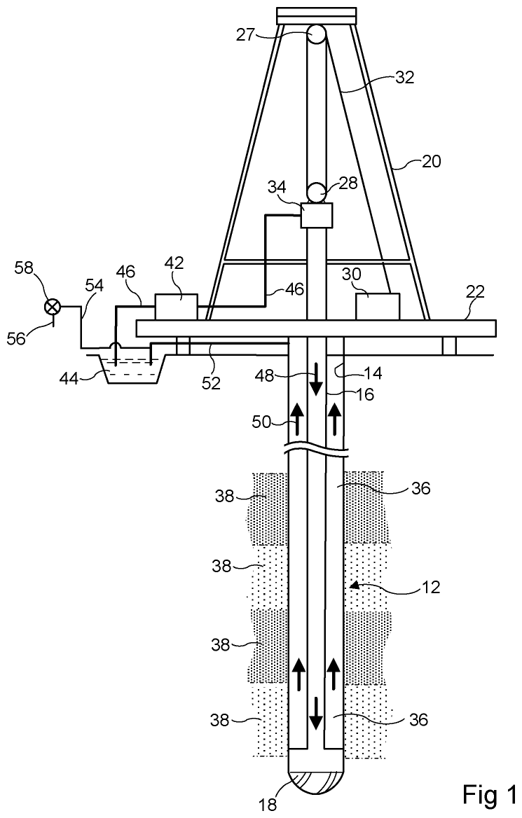

FIG. 1 is a schematic diagram of an example of a conventional drilling system 20 which is shown here to illustrate sampling and testing in accordance with this disclosure. While FIG. 1 illustrates an example of a land-based drilling system, it should be understood that measurement in accordance with the present disclosure can also be used with an offshore drilling system.

As shown, a wellbore 12 bounded by an internal surface 14 is formed in a subterranean formation, and a drill string 16 is suspended within the wellbore 12. At the downhole end of the drill string 16 there is a drill bit 18 that cuts through the subterranean formation as the drill string is rotated. A mast 20 is positioned on a drill floor 22 and over the wellbore 12. A hoisting system includes a crown block 27, a traveling block 28 and a drawworks system 30 with wire rope 32 extending between the crown block 27 and the traveling block 28. In the illustrated embodiment, a top drive 34 is coupled to the traveling block 28. The top drive 34 rotates the drill string 16 as the hoisting system raises and lowers the top drive 34 and the drill string 16 relative to the drill floor 22 to facilitate drilling of the wellbore 12. It should be appreciated that hoisting systems having various other components (e.g., swivels) and/or other configurations may be utilized to drive movement of the drill string 16.

A pump 42 (e.g., piston pump) is configured to pump a drilling fluid into the wellbore 12. As shown, the pump 42 draws a flow of the drilling fluid from the drilling fluid tank 44 and sends it through a fluid conduit 46 (e.g., pipe, line), through a port in the top drive 34, and into an interior channel within the drill string 16. The drilling fluid flows down to the drill bit, as shown by arrow 48 and exits the drill string 16 via ports in the drill bit 18. The fluid then returns upwardly through an annular passage 36 between an outer surface of the drill string 16 and the internal surface 14 that defines the wellbore, as shown by arrows 50. At the surface, the drilling fluid returns to the drilling fluid tank 44 via a fluid conduit 52 (e.g., pipe). The pipe 52 has a branch 54 leading to an outlet 56 where samples of the drilling fluid returning to the surface may be collected by operation of valve 58.

The wellbore 12 extends through various subterranean rock strata 38. These may vary in porosity and where the wellbore extends through porous strata or swellable strata such as clay, some of the drilling fluid or its constituents may enter the strata or some of the liquid naturally present in the strata may enter the drilling fluid flowing towards the surface within the passage 36. Thus, the composition of the aqueous phase of the drilling fluid can change as the fluid circulates, and so it is normal practice to monitor the composition of the drilling fluid returning to the surface by collecting and analyzing samples collected at the outlet 56. If the drilling fluid is formulated to contain chloride, it is a normal requirement to monitor the chloride concentration. In circumstances where a drilling fluid contains bromide rather than chloride, it is normal to monitor the bromide concentration.

A number of experiments were carried out to investigate whether chloride ion concentration can be determined by voltammetry despite variations in constituents other than chloride. The general theory of voltammetry and apparatus for it are covered in standard textbooks, such as Bard and Faulkner “Electrochemical Methods: Fundamentals and Applications” (2nd ed 2001).

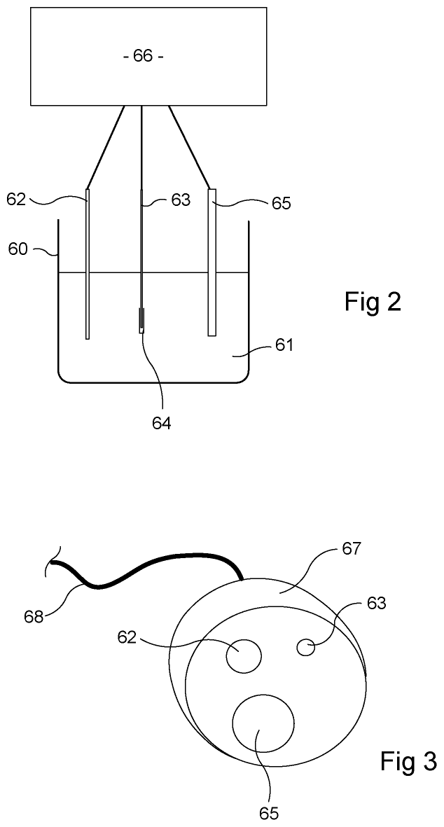

FIG. 2 schematically shows the components of voltammetry apparatus. Three electrodes contact the liquid 61 under test in a container 60. The electrodes are a silver working electrode 62, a reference electrode 63 which may be a standard silver/silver chloride electrode in which a silver electrode is surrounded by a container 64 containing a small quantity of a potassium chloride solution of high ionic strength, and thirdly a counter electrode 65 which may be steel stainless steel, platinum, gold or a carbon-based material such as graphite or glassy carbon. Another possible reference electrode is a sintered silver/silver chloride pellet in an insulated body, available from World Precision Instruments, Sarasota, Florida. The counter electrode 65 has a surface area in contact with the liquid which is at least twice the working electrode's surface area in contact with the liquid. The three electrodes 62, 63, 65 are each electrically connected by wire to a potentiostat 66 which applies a varying potential, relative to the potential of the standard electrode, to the working electrode 62 and records both the applied potential and current flow through the working electrode at that potential.

The electrodes may be attached together in an assembly in which they are insulated from each other. FIG. 3 shows an example of this. The assembly comprises an electrically insulating cylindrical body 67 in which the electrodes are embedded. Each electrode is attached to a wire which runs through the body, enabling connection to a potentiostat. This body 67 may be attached to a flange or other parts so that it can be attached to a container for a liquid sample. The working electrode 62 is a silver disc and may for example have a diameter of 1 cm. The counter electrode 65 may be a platinum disc having a diameter of 2 (mA)cm, so that its exposed area is more than twice the exposed area of the working electrode 62. The reference electrode 63 may be a silver disc on which a deposit of silver chloride is initially formed by connecting the disc to a positive potential and the counter electrode to negative potential while they are both immersed in a sodium chloride solution. Alternatively, the reference electrode may be a solid-state silver/silver chloride pellet. A cable containing the wires for connecting the electrodes 62, 63, 65 to a potentiostat is indicated at 68.

Cyclic voltammetry was employed in these experiments. For this, the potential at the working electrode was varied in one direction over a range and then varied in the reverse direction over the same range. In a first set of experiments, cyclic voltammetry was carried out with aqueous solutions of individual chlorides at various concentrations in demineralized water. In each experiment, the potential of the working electrode was scanned oxidatively (i.e. anodically) from −1.0V to +1.0V at a scan rate of 100 mV per second and then scanned in the reverse direction (reductively, cathodically) at the same scan rate back to the initial potential. On the oxidative sweep, silver ions (Ag⁺) are generated, which combine with the chloride ions (Cl⁻) in solution to form insoluble silver chloride (AgCl), which precipitates out of solution and forms a AgCl layer on the electrode surface, following the reactions below:

Ag→Ag⁺+e⁻

Ag⁺+Cl⁻→AgCl₍ₛ₎

When the potential is swept reductively, the AgCl on the electrode surface is reduced back to Ag and Cl⁻, thus:

AgCl₍ₛ₎+e⁻→Ag+Cl⁻

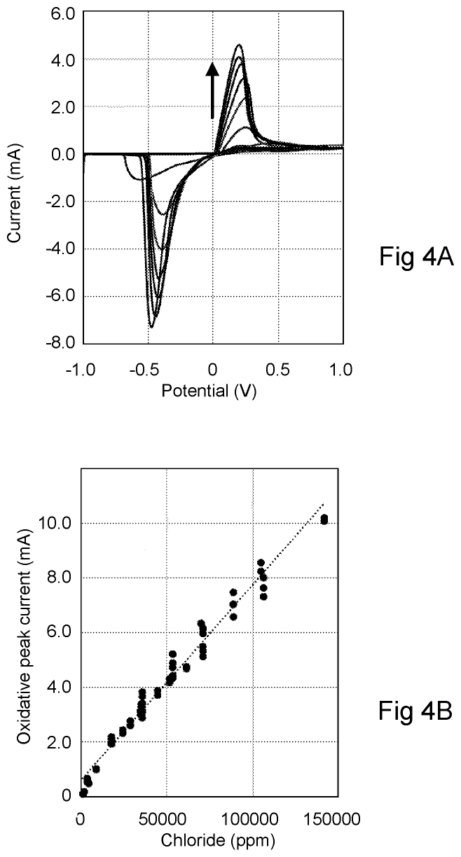

FIG. 4A shows plots of current against potential (referred to as voltammograms) for the above experiments when carried out with sodium chloride solutions. As the chloride concentration increased, the current increased but the potential relative to the reference electrode at which the current reached a peak changed by only a small amount which did not hinder the measurement of peak current. The arrow indicates the direction of increasing chloride concentration and oxidative current. Very similar results were obtained with potassium chloride solutions.

FIG. 4B shows the oxidative peak currents from two repeats of the procedure of FIG. 4A and also includes the oxidative peak currents from similar experiments with potassium chloride in place of sodium chloride, plotted against chloride ion concentration. It was noted that these peak currents were proportional to chloride ion concentration over a range from 1773 ppm (0.5M) to 141800 ppm (4M) chloride. FIG. 4B provides a calibration of the system: within the range where current is proportional to concentration, the concentration of chloride was 14100 ppm per milliamp current.

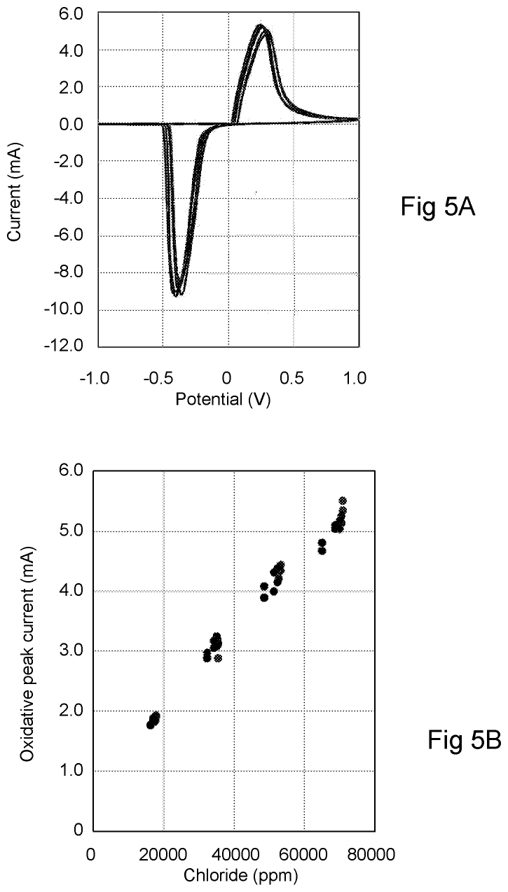

FIG. 5A shows similar plots of current against potential for a 2M potassium chloride solution adjusted to pH values of 6.5, 8, 9, 10 and 10.5 by addition of potassium hydroxide. It was apparent that variation of pH within the range from 6.5 to 10.5 made negligible change to the potential at which current was maximum: the potential for peak current at pH 10.5 differed from that at pH 6 by only 0.025 volt. Similar plots of current against potential were obtained with 0.5M, 1.0M and 1.5M potassium chloride solutions at the same pH values. FIG. 5B shows peak currents plotted against concentration in similar manner to FIG. 4B. Overall, it was apparent that variation of pH within the range from 6.5 to 10.5 did not alter the potential at which current was maximum, neither did it alter the proportionality of peak current to chloride ion concentration.

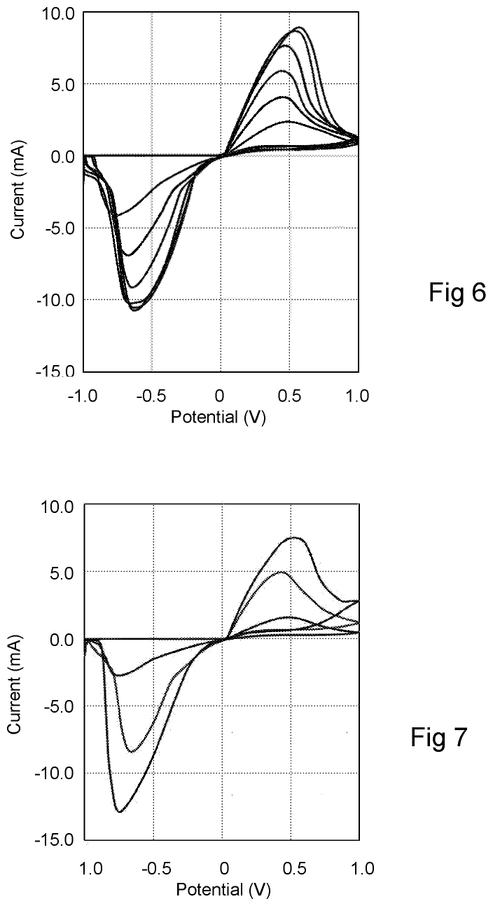

FIG. 6 shows voltammetric plots of current against potential for six solutions containing mixtures of sodium chloride and calcium chloride. The compositions of these solutions were:

| | NaCl | CaCl₂ | total chloride | | Potential for | Peak current |
|---|---|---|---|---|---|---|
| | (ppm) | (ppm) | ppm | moles | peak current (V) | (mA) |
| 1 | 14999 | 0 | 23129 | 0.65 | 0.482 | 2.350 |
| 2 | 15000 | 15003 | 49670 | 1.40 | 0.453 | 4.050 |
| 3 | 15003 | 35000 | 85047 | 2.40 | 0.440 | 5.900 |
| 4 | 15001 | 54995 | 120416 | 3.40 | 0.462 | 7.650 |
| 5 | 14998 | 74993 | 155786 | 4.39 | 0.540 | 8.660 |
| 6 | 14998 | 94990 | 191170 | 5.39 | 0.567 | 8.920 |

The very small change in potential relative to the reference electrode at which peak current was observed did not interfere with measuring the peak current.

FIG. 7 shows plots of current against potential for three solutions containing mixtures of sodium, calcium, magnesium and lithium chlorides. The compositions of the three solutions are given in the following table:

| | | | | | Total chloride | |
|---|---|---|---|---|---|---|
| | Na (ppm) | Ca (ppm) | Li (ppm) | Mg (ppm) | ppm | moles |
| 1A | 40001 | 2000 | 200 | 500 | 67699 | 1.91 |
| 2A | 5000 | 100 | 2000 | 50 | 18249 | 0.51 |
| 3A | 5.2 | 1.2 | 24819 | 1.1 | 126792 | 3.58 |

The ratio of oxidative peak current to total chloride ion concentration was calculated for the various compositions shown in FIGS. 6 and 7. It was found to be approximately the same as the ratio determined with solutions of sodium and potassium chlorides shown in FIG. 4B.

Further experiments were carried out in which mixtures were made containing polymers and clay as in drilling fluids. These were then filtered using API filtration conditions to remove the clay. Then cyclic voltammetry as above was carried out with the filtrates, to discover whether the voltammetric measurements were affected by the dissolved polymers present in the filtrates. The compositions were the same except that sodium hydroxide was added to give various pH values between pH 8 and pH 11.5. The compositions of full formulation fluids, prior to filtration, were:

| | | |
|---|---|---|
| Xanthan viscosifier (Duo-Vis) | 1.5 | g/L |
| Carboxymethyl cellulose (PolyPAC) | 0.429 | g/L, |
| Carboxymethyl cellulose (PolyPAC ELU) | 1.071 | g/L, |
| Preservative (Safecide) | 1 | g/L, |
| Bentonite clay | 10 | g/L, |
| Potassium chloride | 75 | g/L, |
| Barium sulfate (Barite) | 200 | g/L, |
| Sodium hydroxide (to vary pH). | | |

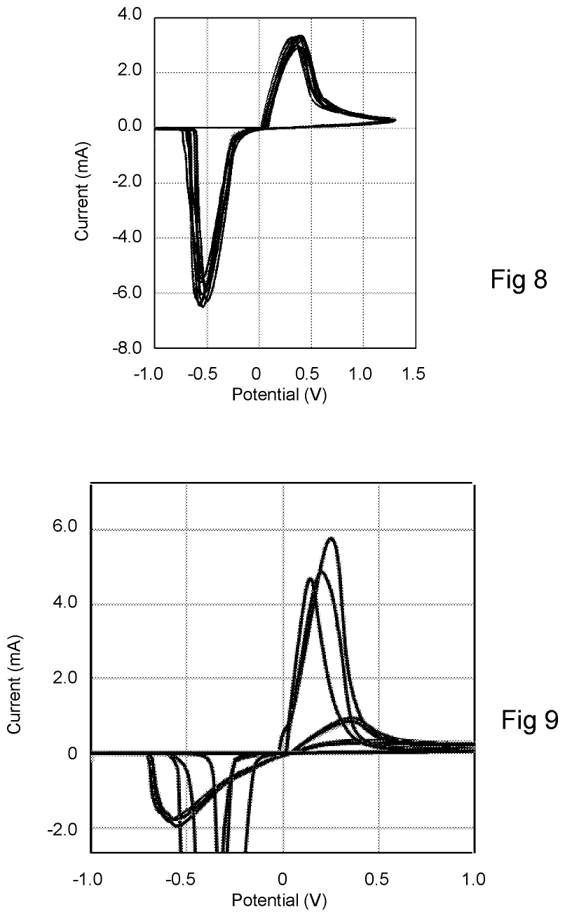

The voltammograms are shown superimposed in FIG. 8. As can be seen, they were very similar, despite the variation in pH, and were very similar to the voltammograms shown in FIGS. 4A and 5A indicating that the presence of polymers in the filtrate was not affecting the electrochemical measurements.

In a further experiment, sample compositions were prepared, exemplifying compositions containing a mixture of salts as might occur in water produced from a subterranean location. The compositions are set out in the following table:

| | Na | K | Ca | Mg | Cl | Br | SO₄ | Ba | Sr | Total |
|---|---|---|---|---|---|---|---|---|---|---|
| | (ppm) | (ppm) | (ppm) | (ppm) | (ppm) | (ppm) | (ppm) | (ppm) | (ppm) | (ppm) |
| 1 | 51610 | 10758 | 1177 | 10219 | 118724 | 10298 | | 120 | 251 | 201047 |
| 2 | 4019 | 0 | 1133 | 71 | 8597 | 0 | 0 | 104 | 176 | 14100 |
| 3 | 4109 | 0 | 311 | 0 | 7064 | 0 | 0 | 120 | 154 | 11857 |
| 4 | 0 | 0 | 4065 | 0 | 8437 | 0 | 0 | 800 | 1028 | 14331 |
| 5 | 80848 | 1881 | 0 | 3463 | 135483 | 941 | 3363 | 0 | 0 | 227157 |

FIG. 9 shows the oxidative part of the voltammograms for these solutions and also for a 3M potassium chloride solution in demineralised water. It can be seen that the potentials for oxidative peak current are similar, even though there are differences in composition.

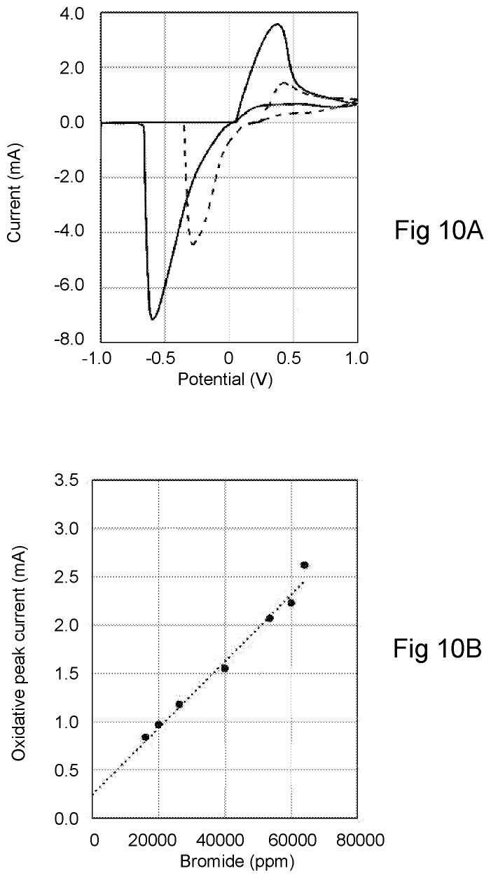

In some circumstances, drilling fluids are formulated with sodium or potassium bromide rather than chloride. A silver electrode can detect bromide through the formation of insoluble silver bromide, and voltammetry shows a peak current for bromide at a potential which is close to the peak potential for chloride. This is shown by FIG. 10A in which a voltammogram for sodium bromide is shown as a continuous line and a voltammogram for sodium chloride is shown with a dashed line. Cyclic voltammetry as described above with reference to FIGS. 4A and 4B was carried out using sodium bromide rather than sodium chloride. FIG. 10B shows the peak current plotted against bromide concentration. There is a range of concentration over which the peak current was proportional to bromide concentration.

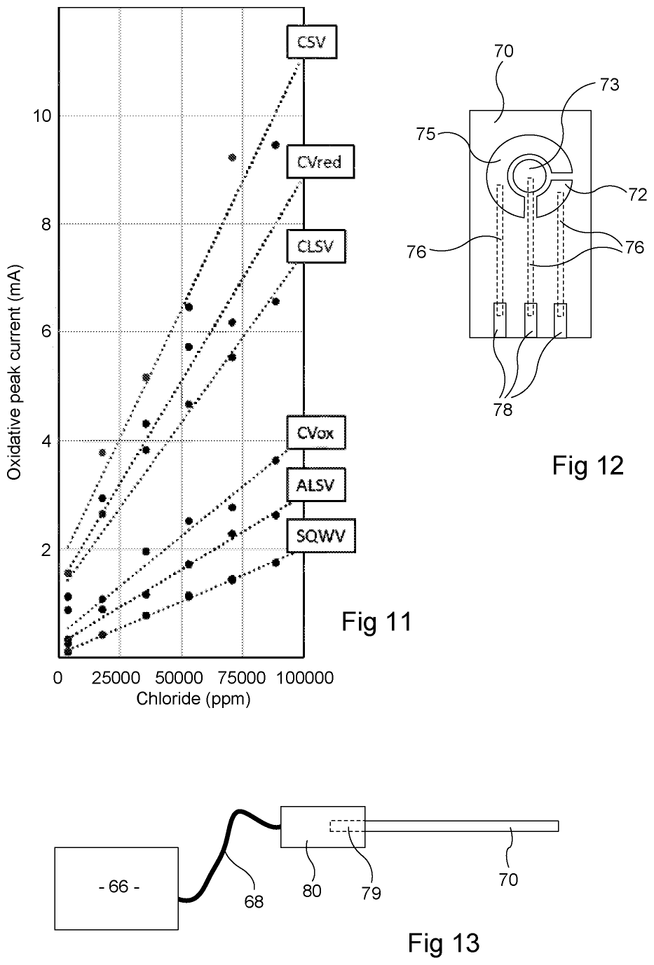

The experiments above were all carried out using cyclic voltammetry, with the results taken from the oxidative peak. However, other voltammetric techniques can be used, as demonstrated by the following experiment. A stock solution containing 2.5M KCl in demineralized water was diluted to provide solutions of various concentrations. The apparatus described above was operated to carry out cyclic voltammetry, with results taken from either the oxidative or the reductive peak, as well as anodic and cathodic linear sweep voltammetry and cathodic stripping voltammetry. In each case, the applied potential was varied at a scan rate of 20 mV/s. For cathodic stripping voltammetry the electrode was pretreated by applying potential of +1V relative to the Ag/AgCl reference electrode for 180 seconds in 2.5M KCl. The apparatus was also operated to carry out square wave voltammetry with parameters of 2 mV for step potential, 20 mV for amplitude and 25 Hz for frequency. All of these techniques showed peak currents which were proportional to chloride concentration over a range of concentrations, as shown by FIG. 11.

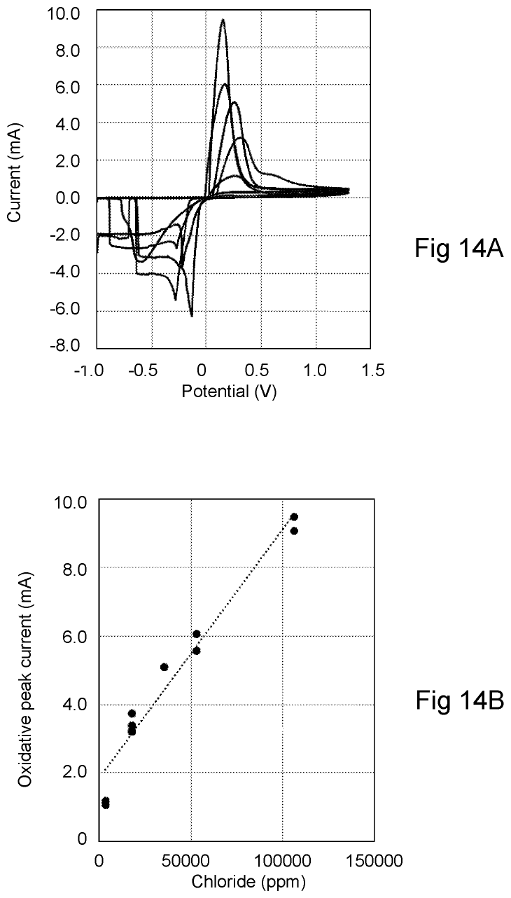

FIGS. 12 and 13 show a different electrode assembly which may be smaller than that shown in FIG. 3, which may require only a small volume of liquid, and which may be manufactured as a disposable item. It has a laminar ceramic base 70 onto which a working electrode 72, reference electrode 73 and counter electrode 75 are screen printed. These electrodes are positioned to lie over and be electrically connected to end portions of three previously printed conductive tracks 76 which connect the electrodes to contact areas 78. An electrically insulating layer is applied over the parts of the conductive tracks 76 between the electrodes 72, 73, 75 and the contact areas 78. As shown in FIG. 13, the end portion 79 of the ceramic base 70 which carries the contact areas 78 can be pushed into a connector 80 so as to project horizontally from it. The connector 80 has spring contacts to connect with the areas 78 and these contacts are connected by wires running within cable 68 to a potentiostat 66. When such an electrode assembly 70 is held in such a horizontal position by a connector 80, voltammetry can be carried out using a few drops (typically less than 0.5 ml) of liquid placed on the electrodes 72,73,75. FIG. 14A shows voltammograms obtained with such an electrode assembly and FIG. 14B is a plot of peak current against potential, showing that peak current was proportional to chloride ion concentration over a range from about 2000 ppm to 100,000 ppm.

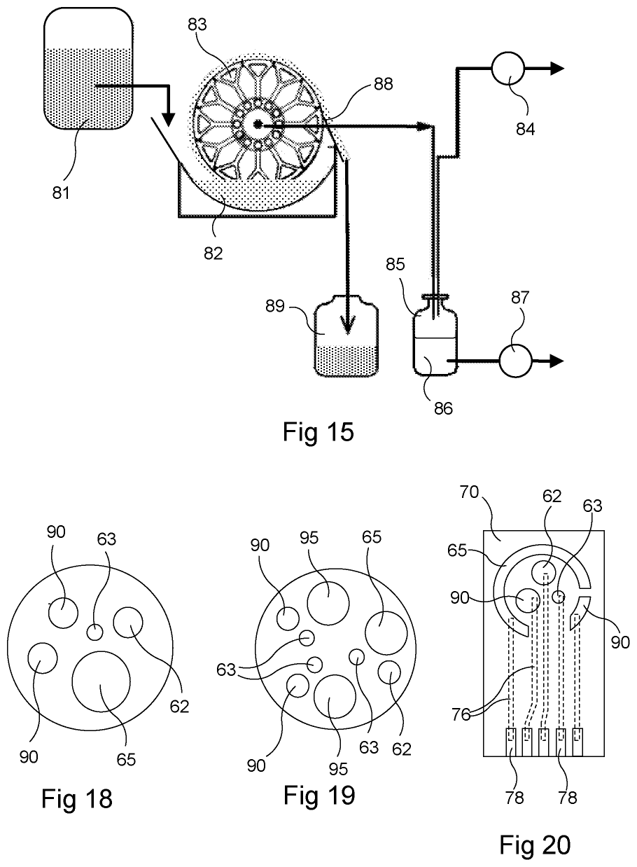

A procedure for analytical processing of collected samples is shown by FIG. 15. As a first step after collecting a sample, for instance a sample from the outlet 56 of FIG. 1, the solids in the sample are removed. This may be done by filtration, in accordance with API standard procedure, but a faster method is to use a rotary vacuum drum as diagrammatically illustrated by FIG. 15. A collected sample 81 is poured into a tray 82 below a rotating drum 83 which has a porous filter material around its circumference. Vacuum applied to the drum interior by a vacuum pump 84 draws the liquid part of the sample through the filter material into the drum interior and from there to a trap 85 in which the liquid 86 collects until it is drawn out by a filtrate pump 87 or collected manually after filtration of the sample is complete. Solids collecting on the filter material; around the drum circumference are scraped off at 88 and fall into a receptacle 89.

A measured amount of a demulsifying surfactant, which is not containing chloride or bromide ions, is mixed with a measured volume of the liquid part 86 so that the aqueous and oil phases in the sample separate from each other. A quantity of the aqueous phase is next examined by voltammetry to determine chloride ion and/or bromide ion concentration.

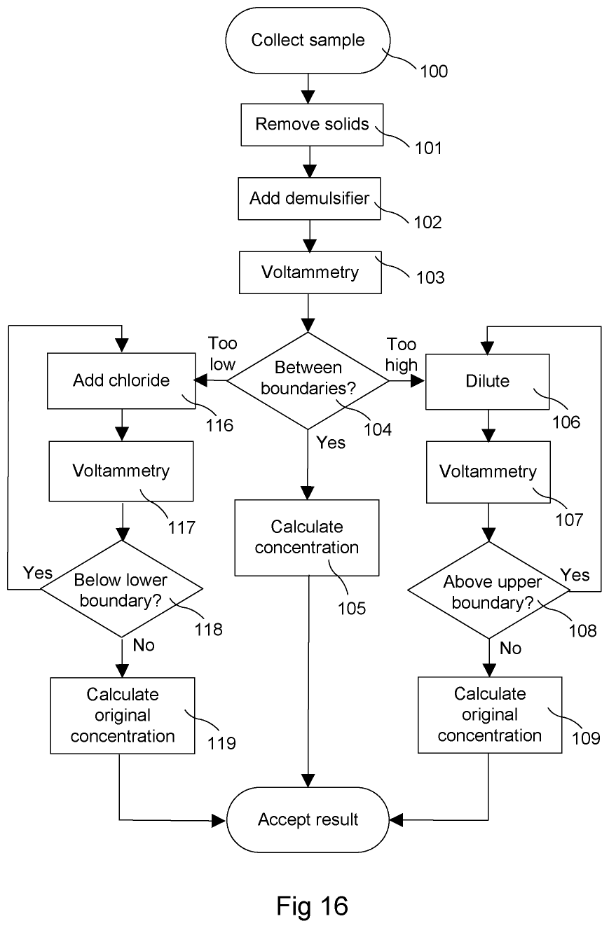

The experimental work described above with reference to FIGS. 4 to 10 has shown that chloride concentration is proportional to peak current over a range of concentrations. However, this proportional relationship between peak current and concentration will not hold at low chloride concentrations nor at high chloride concentrations. FIG. 16 is a flow chart showing a procedure for voltammetry which extends the range of concentrations which can be measured. To simplify explanation, it is assumed that bromide is not present. The first step 100 shown in FIG. 16 is the collection of a sample, for instance collection at outlet 56 as shown in FIG. 1. The following step 101 is removal of solid constituents of the sample, which may be achieved by filtration in accordance with API procedure, or by using a vacuum drum as in FIG. 16. Step 102 is the addition of demulsifying surfactant so that the aqueous and oil phases become separate layers.

The next step 103 is to carry out voltammetry with some of the aqueous liquid part of the sample and decide (indicated at 104) whether the measured peak current is above an upper boundary or below a lower boundary of a range in which concentration is proportional to peak current. If the current is within this range, the concentration of chloride is calculated from the peak current, as indicated at 105.

If the voltammetry observes a peak current which is above the upper boundary, the following step 106 is to dilute a measured volume of the sample liquid with a measured amount of water and then at 107 carry out voltammetry and at 108 decide whether the peak current measured with the diluted sample is again above the upper boundary If the peak current measured with this diluted sample is below the upper boundary (but not so low as to be below the lower boundary), the concentration of chloride in the liquid of the original sample is calculated at 109 from the result of voltammetry at 107 and the amount of dilution. If the peak current measured with the concentration at 107 is again above the upper boundary, steps 106 to 108 are repeated with greater dilution.

If voltammetry at 104 on the initial liquid part of the original sample shows a peak current below the lower boundary, the next step 116 is to add a known amount of chloride to a measured volume of the initial sample liquid to raise the chloride concentration and then at 117 carry out voltammetry to determine peak current in this more concentrated liquid. If the peak current, then corresponds to a concentration above the lower boundary, as indicated at 118, the concentration of chloride in the liquid of the original sample is calculated at 119, taking account of the amount of chloride added to raise the chloride concentration. If the peak current measured at 117 is still not above the lower boundary, step 116 is repeated with a greater amount of added chloride.

In view of the experimental work described above, the upper boundary may be chosen as 7 mA corresponding to a chloride concentration slightly below 100,000 ppm of chloride and the lower boundary may be chosen as 0.2 mA corresponding to a chloride concentration slightly above 2500 ppm of chloride. It is not essential that the upper and lower boundaries are at the extremities of the range over which peak current is proportional to chloride concentration, but they should be chosen so that peak current is proportional to chloride concentration throughout the range between the chosen lower and upper boundaries.

An operator carrying out determination of chloride concentration may make choices as to the values to use as boundaries and what action to take if the current/chloride concentration determined at step 104 is outside one or other of the boundaries.

For example, an operator might choose to adopt a smaller range between boundaries, such as 0.5 mA as lower boundary and 5 mA as upper boundary. If a sample was found to show a peak current of 0.6 mA at step 104 and then diluted with an equal volume of deionized water at step 106, a peak current of 0.3 mA at step 107 would confirm that a concentration calculated from the value of 0.6 mA at step 104 was correct.

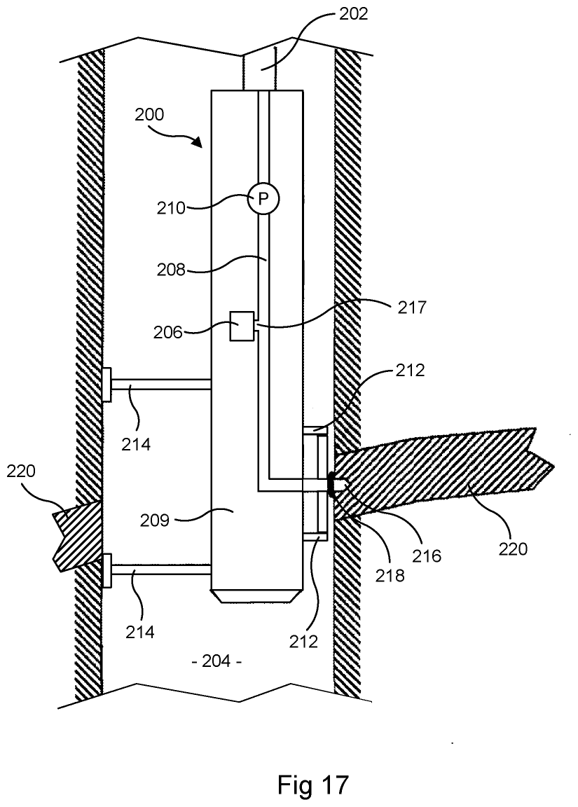

FIG. 17 shows a formation testing apparatus 200 held on a wireline 202 within a wellbore 204. This testing apparatus is used here for collecting samples of the water in porous rock surrounding the wellbore 204 and determining the chloride ion concentration in the collected water. The apparatus 200 may be part of a modular dynamic tester as described in U.S. Pat. No. 3,859,851 to Urbanosky, U.S. Pat. No. 3,780,575 to Urbanosky and U.S. Pat. No. 4,994,671 to Safinya et al., with this known tester being modified by introduction of an electrochemical analyzing sensor 206 which includes an assembly with three electrodes as shown in FIG. 3. The modular dynamics tester comprises body 209 containing a main flowline 208. The analyzing sensor 206 is positioned so that the three electrodes are exposed to liquid in the flowline 208 via opening 217. The flow through the flowline 208 is driven by means of a pump 210 located towards the upper end of the flowline 208. The body 209 is equipped with hydraulically extendable arms 212 and counterarms 214. The arms 214 carry a sample probe tip 216 for sampling fluid. The base of the probing tip 216 is isolated from the wellbore 204 by an O-ring 218, or other sealing devices.

Before completion of a well, the modular dynamics tester 200 is lowered into the well on the wireline 202. After reaching a target depth so as to be adjacent to the layer 220 of the surrounding formation, which is to be sampled, the hydraulic arms 212 and counter arms 214 are extended to hold the testing apparatus 200 in position and project the sample probe tip 216 into the formation. The O-ring 218 at the base of the sample probe 216 forms a seal between the interior of the wellbore 204 and the formation layer 220 into which the probe 216 is inserted and prevents the sample probe 216 from acquiring fluid directly from the borehole 204.

Once the sample probe 216 is inserted into the formation layer 220, an electrical signal is passed down the wireline 202 from the surface so as to start the pump 210 and the sensor system 206 to begin sampling of a sample of fluid from the formation layer 220. The electrochemical sensor 206 can then measure the chloride ion content of the liquid drawn from the formation by the pump 210.

FIGS. 18, 19 and 20 show further possibilities for electrode assemblies. FIG. 18 shows an electrode assembly comprising a cylindrical insulating body with a silver working electrode 62, an Ag/AgCl reference electrode 63 and a platinum counter electrode 65 as shown by FIG. 20. In addition, it has one or more further working electrodes 90 which are not silver and do not detect chloride ions. Each additional working electrode is used for measuring another ion, where it is possible to use the same reference and counter electrodes. This arrangement enables voltammetry for two or more ions to be carried out consecutively or perhaps concurrently, using the same apparatus and liquid sample.

For example, one electrode 90 may be edge-plane pyrolytic graphite for voltammetric determination of hydrogen ion concentration (i.e. pH) and the other working electrode 90 may be boron-doped diamond, used for determination of hydrogen sulphide concentration.

As shown by FIG. 19, this approach may also be implemented with a separate reference electrode and counter electrode 95 associated with each of the working electrodes 90.

Such provision of electrodes for more than one analyte may also be implemented with electrodes deposited on a flat ceramic element as shown by FIG. 20, where a single counter electrode 65 is shaped as an arc, working 62 and reference 63 electrodes are circular, one additional working electrode 90 is circular and the other is an arc. All five electrodes are connected to respective contact areas 78 in a similar manner to that described with reference to FIG. 12.

An assembly with electrodes for more than one analyte may in particular be used as the sensor 206 in the testing apparatus 200 shown in FIG. 17, enabling that apparatus to determine the concentrations of chloride and/or bromide and also the concentrations of other ions in the flow drawn from the formation layer 220 and passing along the flowline 208.

Although only a few examples have been described in detail above, those skilled in the art will readily appreciate that many modifications are possible in the examples without materially departing from this subject disclosure. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

1. A method of determining a concentration of analyte ions, which are chloride ions or bromide ions, in an aqueous fluid at a subterranean location or brought to the surface from a subterranean location, the method comprising:
providing a voltammetric system comprising a working electrode, a reference electrode, a counter electrode, and electronic circuitry configured to apply a varying potential, relative to the reference electrode, to the working electrode and to measure and record the current through the working electrode;
contacting a portion of the aqueous fluid with the electrodes, wherein the counter electrode's surface area exposed to the fluid is at least twice the working electrode's surface area exposed to the fluid; and
operating the voltammetric system to apply a potential to the working electrode across a range in which the applied potential causes an electrochemical reaction in which chloride ions are liberated into or removed from the solution at the working electrode, and the current for that reaction passes through a peak value, and recording the current at the peak, or recording the current over a range of potentials including the peak, as indicative of the concentration of the analyte ions in the fluid.

2. The method of claim 1, further comprising collecting a sample containing the aqueous fluid and suspended solids, and separating the aqueous fluid from the solids before contacting a portion of the aqueous fluid with the electrodes.

3. The method of claim 2, wherein separating the aqueous fluid from the solids comprises placing the sample in contact with the exterior of a rotary drum having a filter material at its surface, and rotating the drum while pumping air from the interior of the drum, so that the liquid part of the sample passes through the filter material into the drum while the solid part cannot pass through the filter material.

4. The method of claim 2, wherein the sample contains an oil phase, and the method comprises separating the aqueous solution from the oil phase before contacting a portion of the aqueous fluid with the electrodes.

5. The method of claim 2, wherein the aqueous solution is the aqueous phase of a drilling fluid containing a continuous aqueous phase and a discontinuous oil phase, with at least one chloride or bromide salt dissolved in the aqueous phase, a viscosity enhancing polymer in the aqueous phase, and insoluble particulate material suspended in the aqueous phase.

6. The method of claim 1, wherein the potential is applied to the working electrode over a range extending at least 0.5 volt below and at least 0.5 volt above the peak current potential.

7. The method of claim 1, wherein the potential is applied to the working electrode over a range that extends no more than 1.5 volt above and no more than 1.5 volt below the potential at which the current reaches a peak.

8. The method of claim 1, further comprising determining whether the peak current exceeds a predetermined value; and, if so, mixing a known amount of diluent with a known amount of the sample to form a diluted sample, contacting the diluted sample with the electrodes, and again operating the voltammetric system to apply potential to the working electrode across the range and recording the current at the peak, or over a range of potential including the peak, as indicative of the concentration of the analyte ions in the diluted sample.

9. The method of claim 1, further comprising determining whether the peak current is less than a predetermined value; and, if so, adding a known amount of additional analyte to a known amount of the sample to form a more concentrated sample, contacting the more concentrated sample with the electrodes, and again operating the voltammetric system to apply potential to the working electrode across the range, and recording the current at the peak, or over a range of potential including the peak, as indicative of the concentration of analyte ions in the more concentrated sample.

10. The method of claim 1, wherein the working electrode, the reference electrode, and the counter electrode are attached together, but are electrically insulated from each other, as an electrode assembly.

11. The method of claim 10, wherein the electrode assembly comprises a laminar base with the working electrode, reference electrode, and the counter electrode formed as deposits on separate areas of the laminar base.
